(19) United States
(12) Patent Application Publication
(10) Pub. No.: US 2018/0280180 A1
(43) Pub. Date: Oct. 4, 2018

(54) JOINT MECHANISM

(71) Applicant: HONDA MOTOR CO., LTD., Tokyo (JP)

(72) Inventor: Tomoya Amari, Wako-shi (JP)

(21) Appl. No.: 15/937,909

(22) Filed: Mar. 28, 2018

(30) Foreign Application Priority Data
Mar. 31, 2017 (JP) 2017-069857

(51) Int. Cl. A61F 5/01

(52) U.S. Cl. CPC A61F 5/0123; A61F 2005/0165; A61F 2005/0158

(57) ABSTRACT

A joint mechanism pivotably connecting first and second links respectively to be fitted on first and second body parts of a user which are connected to each other via a joint, the joint mechanism including: a first slide member connected to the first link and provided with a first slide track having an arc shape that is convex away from the joint in side view; an intermediate slide member provided with a second slide track having an arc shape that is convex away from the joint in side view, and connected to the first slide member to be slidable along the first slide track; and a second slide member connected to the intermediate slide member to be slidable along the second slide track, and connected to the second link, wherein the first and second slide tracks track have different centers of curvature.

### TECHNICAL FIELD

The present invention relates to a joint mechanism to be worn on a joint of a user to assist motion of a body of the user.

### BACKGROUND ART

A conventional leg orthosis for assisting motion of a leg of a user disclosed in JP2012-90758A includes a thigh link to be attached to a thigh, a lower leg link to be attached to a lower leg, and a foot link to be attached to a foot. In the leg orthosis disclosed in JP2012-90758A, the lower leg link includes a U-shaped upper frame, a U-shaped lower frame, and an intermediate frame. Each of the ends of the U-shaped upper frame of the lower leg link is rotatably connected to the thigh link at a corresponding side of the knee joint, and a substantially central part of the upper frame in the lengthwise direction thereof is located in front of and obliquely below the knee. Each of the ends of the U-shaped lower frame is rotatably connected to the foot link at a corresponding side of the ankle joint, and a substantially central part of the lower frame in the lengthwise direction thereof is positioned in front of and obliquely above the ankle. The intermediate frame has an upper end connected to the upper frame at a position in front of and obliquely below the knee, and a lower end connected to the lower frame at a position in front of and obliquely above the ankle. In this leg orthosis, to allow the leg orthosis to be used irrespective of individual differences in muscle condition at the calf portion and to prevent the leg orthosis from hindering the motion of the leg other than that on which the leg orthosis is fitted during walking or any other motion, the lower leg link is configured to be disposed in front of the shin of the user, and the leg orthosis has no structural component to be placed on an inner side of the shin.

Incidentally, a knee joint constituted by a femur and a tibia (knee joint in the narrow sense; namely, tibiofemoral joint) is often considered a hinge joint for simplicity but is actually a helical joint, and may undergo flexing/extending motion (bending/stretching motion) and rotational motion. The flexing/extending motion is a combination of rolling motion, in which the femur rolls on the tibia, and sliding motion, in which the femur slides on the tibia. Therefore, the position of the axis of motion of the knee joint moves depending on the joint angle (bending angle).

However, in the conventional leg orthosis, the thigh link and the lower leg link are rotatably connected to each other by a pair of joints disposed at either lateral side of the knee joint of the user such that the thigh link and the lower leg link are rotatable (pivotable) about a fixed axis determined by the pair of joints. As a result, depending on the joint angle, the position of the pair of joints (and hence, the axis of relative rotation of the thigh link and the lower leg link) may deviate from the axis of motion of the knee joint, causing discomfort to the user when flexing/extending motion takes place.

### SUMMARY OF THE INVENTION

In view of such problems of the prior art, a primary object of the present invention is to provide a joint mechanism of a motion assist device that can reduce the discomfort caused to the user of the motion assist device during motion.

To achieve such an object, one aspect of the present invention provides a joint mechanism (24) pivotably connecting a first link (21) and a second link (22) respectively to be fitted on a first body part (17) and a second body part (18) of a user (U) to assist motion of the user, the first and second body parts being connected to each other via a joint (1), the first link extending along an extension side of the first body part when fitted on the first body part, the second link extending along an extension side of the second body part when fitted on the second body part, the joint mechanism comprising: a first slide member (33) connected to the first link and provided with a first slide track (33b) having an arc shape that is convex away from the joint in side view; an intermediate slide member (40) provided with a first corresponding slide track (41b) configured to cooperate with the first slide track and a second slide track (42c) having an arc shape that is convex away from the joint in side view, and connected to the first slide member so as to be slidable along the first slide track; and a second slide member (37) provided with a second corresponding slide track (37b) configured to cooperate with the second slide track, connected to the intermediate slide member so as to be slidable along the second slide track, and connected to the second link, wherein the first and second slide tracks have different centers of curvature (C1, C2) from each other.

According to this arrangement, because first slide track, along which the intermediate slide member is slidable relative to the first slide member, and the second slide track, along which the second slide member is slidable relative to the intermediate slide member, have different centers of curvature, the instant center of rotation of the second slide member relative to the first slide member is located on a line segment that connects the center of curvature of the first slide track and the center of curvature of the second slide track to each other. As the intermediate slide member slides along the first slide track (namely, pivots about the center of curvature of the first slide track), the line segment rotates about the center of curvature of the first slide track together with the center of curvature of the second slide track, to define a fan-shaped area (A2), in which the instant center of rotation of the second slide member may be located. In other words, the instant center of rotation of the second link (which is connected to the second slide member) relative to the first link is moveable within the fan-shaped area, and this reduces the discomfort caused to the user when the joint is moved.

In the above arrangement, preferably, the intermediate slide member (40) includes: a first intermediate slide member (41) provided with the first corresponding slide track (41b) and a third slide track (41c) having an arc shape that is convex away from the joint in side view, and connected to the first slide member (33) so as to be slidable along the first slide track (33b); and a second intermediate slide member (42) provided with the second slide track (42c) and a third corresponding slide track (42b) configured to cooperate with the third slide track, and connected to the first intermediate slide member so as to be slidable along the third slide track, wherein the third slide track has a center of curvature (C3) different from the centers of curvature of the first slide track and the second slide track.

According to this arrangement, it is possible to widen the area in which the instant center of rotation of the second slide member relative to the first slide member may be located.

In the above arrangement, preferably, the second slide track (42c) has a radius of curvature (R2) different from a radius of curvature (R1) of the first slide track (33b).

According to this arrangement, the length of the line segment connecting the center of curvature of the first slide track and the center of curvature of the second slide track to each other can be adjusted as desired, whereby the fan-shaped area defined by rotation of the line segment can be given a desired size.

In the above arrangement, preferably, the third slide track (41c) has a radius of curvature (R3) different from at least one of a radius of curvature (R1) of the first slide track (33b) and a radius of curvature (R2) of the second slide track (42c).

According to this arrangement, the length of the line segment connecting the center of curvature of the first slide track and the center of curvature of the second slide track to each other can be adjusted as desired, whereby the fan-shaped area defined by rotation of the line segment can be given a desired size.

In the above arrangement, preferably, at least one of the first slide member (33) and the second slide member (37) is connected to a corresponding one of the first link (21) and the second link (22) so as to be pivotable about a pivot axis to be parallel with an axis of motion (1X) of the joint (1).

According to this arrangement, the relative angle between the first link and the second link can be adjusted to be in conformity with the angle of the joint of the user in a basic (natural) posture.

In the above arrangement, preferably, the joint mechanism further includes an elastic member (45) connected to the first link (21) and one of the second link (22) and the second slide member (37) such that, when expanded, the elastic member produces a moment on the first link and the second link in a direction for extending the joint.

According to this arrangement, a moment (tensile force) for extending the joint (or for bringing the first link and the second link closer to each other) can be produced with a simple structure.

In the above arrangement, preferably, the joint mechanism further includes a moment adjustment mechanism (57) configured to adjust the moment produced from the elastic member (45) by adjusting a connection position of the elastic member to the first link (21) in a direction of expansion of the elastic member.

According to this arrangement, an amount of assisting moment appropriate to each user can be applied on the first link and the second link.

In the above arrangement, preferably, the first body part is a thigh (17), the second body part is a lower leg (18), and the joint is a knee joint (1), wherein the first slide member (33), the intermediate slide member (40), and the second slide member (37) are configured to be arranged in front of the knee joint.

According to this arrangement, when the user flexes/extends the knee joint, the instant center of rotation of the second slide member relative to the first slide member can move in accordance with movement of the axis of motion of the knee joint, and therefore, the discomfort caused to the user during flexing/extending motion of the knee joint can be reduced.

In the above arrangement, preferably, the first and second slide tracks are configured such that an area (A) in which an instant center of rotation of the second slide member (37) relative to the first slide member (33) may be located includes a trajectory of an axis of motion (1X) of the knee joint (1).

According to this arrangement, it is ensured that the knee joint mechanism can follow the movement of the axis of motion of the knee joint of the user to thereby reduce the discomfort caused to the user during the flexing/extending motion of the knee joint. It is also possible to define the area in which the instant center of rotation of the second slide member relative to the first slide member may be located such that the area includes trajectories of the axis of motion of the knee joint of a plurality of users, such that the knee joint mechanism can be commonly used by the plurality of users.

### EFFECT OF THE INVENTION

Thus, according to an aspect of the present invention, there is provided a joint mechanism that can reduce the discomfort caused to the user during motion.

### BRIEF DESCRIPTION OF THE DRAWINGS

FIG. 1 is a perspective view of a knee joint of a human right leg;

FIG. 2 is a sectional view of the knee joint of the human right leg along the sagittal plane;

FIG. 3 is a perspective view of a right leg portion of a motion assist device employing a knee joint mechanism according to an embodiment of the present invention, where the knee joint mechanism is shown in a contracted state corresponding to an extended state of the knee joint;

FIG. 4 is an exploded perspective view of the knee joint mechanism shown in FIG. 3;

FIG. 5 is a sectional view taken along line V-V in FIG. 3;

FIG. 6 is a sectional view taken along line VI-VI in FIG. 3;

FIG. 7 is a schematic inner side view of the knee joint of the right leg showing an area in which an instant center of rotation of the knee joint mechanism may be present; and

FIG. 8 is a sectional view similar to FIG. 6 and showing the knee joint mechanism in a deployed state corresponding to a flexed state of the knee joint.

### DESCRIPTION OF THE PREFERRED EMBODIMENT(S)

In the following, preferred embodiments of the present invention will be described in detail with reference to the appended drawings.

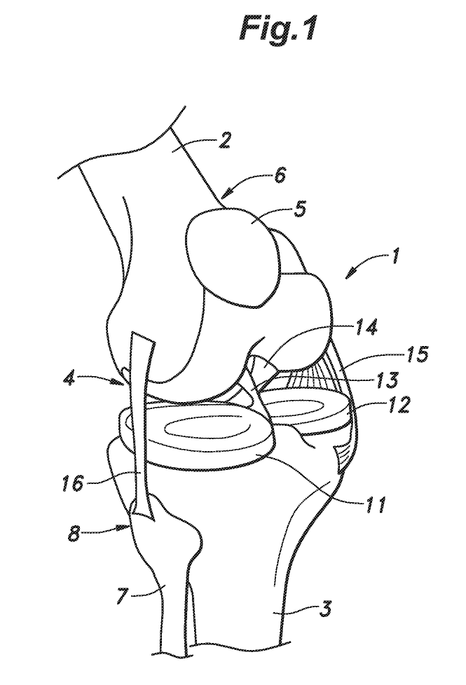

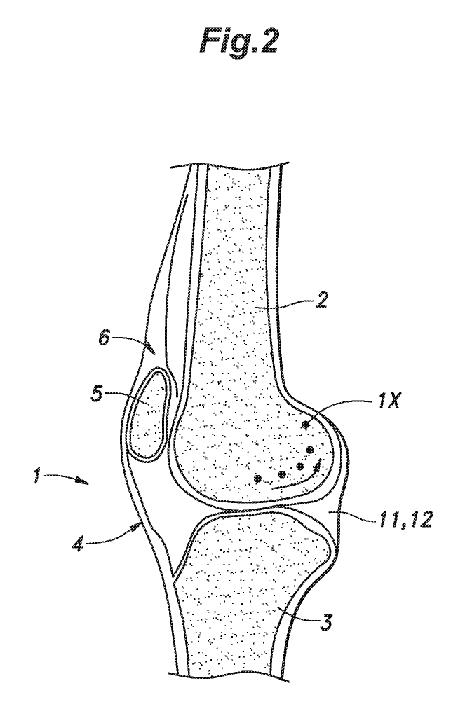

First, a human knee joint 1 will be described with reference to FIGS. 1 and 2. FIG. 1 is a perspective view of the human knee joint 1. The knee joint 1, in a broad sense, refers to a multi-joint including a tibiofemoral joint 4 constituted by a femur 2 and a tibia 3 and a patellofemoral joint 6 constituted by the femur 2 and a patella 5. In a boarder sense, the knee joint 1 may further include a tibiofibular joint 8 constituted by the tibia 3 and a fibula 7. In the narrow sense, the knee joint 1 refers to the tibiofemoral joint 4. In this description, the term “knee joint 1” is used in the narrow sense to refer to the tibiofemoral joint 4.

The knee joint 1 is a helical joint, though often regarded as a hinge joint for simplicity, capable of performing flexing/extending motion and rotational motion, and has a structure in which the lateral and medial condyles of the femur 2 at the lower end thereof respectively contact the lateral and medial condyles of the tibia 3 at the upper end thereof via lateral and medial menisci 11, 12, etc. The knee joint 1 is stabilized by an anterior cruciate ligament 13, a posterior cruciate ligament 14, a medial collateral ligament 15, a lateral collateral ligament 16, etc. surrounding the knee joint 1. Rotational motion of the knee joint 1 is smaller than flexing/extending motion of the same, and in the present description, motion of the knee joint 1 is regarded as flexing/extending motion consisting of flexion and extension, and an axis of motion 1X (see FIG. 2) of the knee joint 1 is regarded as extending laterally (along the frontal plane).

When the knee joint 1 undergoes a flexing motion, with the femur 2 moved relative to the tibia 3 in a fixed state, rearward rolling and forward sliding of the femoral condyles on the tibial condyles take place simultaneously. During an initial stage of flexion, the femoral condyles undergo essentially rearward rolling only, and as the flexion advances, the ratio of forward sliding increases. Thereafter, the forward sliding is terminated and the femoral condyles roll there. In an extending motion of the knee joint 1, the femoral condyles undergo the above process in the reverse order. As a result of the above motion of the knee joint 1, if the knee joint 1 undergoes a flexing motion with the tibia 3 moved relative to the femur 2 in a fixed state, the axis of motion 1X of the knee joint 1 moves as explained below.

FIG. 2 is a sectional view showing the knee joint 1 of the human right leg along the sagittal plane. As shown in FIG. 2, when the knee joint 1 is in an extended state (or in an initial stage of flexion), the axis of motion 1X of the knee joint 1 is located at a substantially central part of a lower end portion of the femoral condyle in the fore-and-aft direction. As the knee joint 1 flexes and the joint angle (bending angle) of the knee joint 1 increases (here, the joint angle of the knee joint 1 is defined to increase as the knee joint 1 flexes from the extended state), the axis of motion 1X of the knee joint 1 moves rearward and thereafter upward as shown by dots and an arrow in FIG. 2. On the other hand, in the extending motion of the knee joint 1 from the flexed state, the axis of motion 1X of the knee joint 1 moves in a reverse manner; namely, as the joint angle decreases the axis of motion 1X of the knee joint 1 moves downward from a rear end portion of the femoral condyle, and then, moves forward.

Thus, to follow such movement of the axis of motion 1X of the knee joint 1, a knee joint mechanism 24 of the present embodiment is configured such that the pivot axis thereof is movable. In the following, a motion assist device 20 provided with the knee joint mechanism 24 will be described in detail. In the following description, the limb position when the user is standing upright is referred to as a basic limb position, and the structure of the motion assist device 20 will be described, assuming that the limb of the user U having the motion assist device 20 fitted thereon is in the basic limb position.

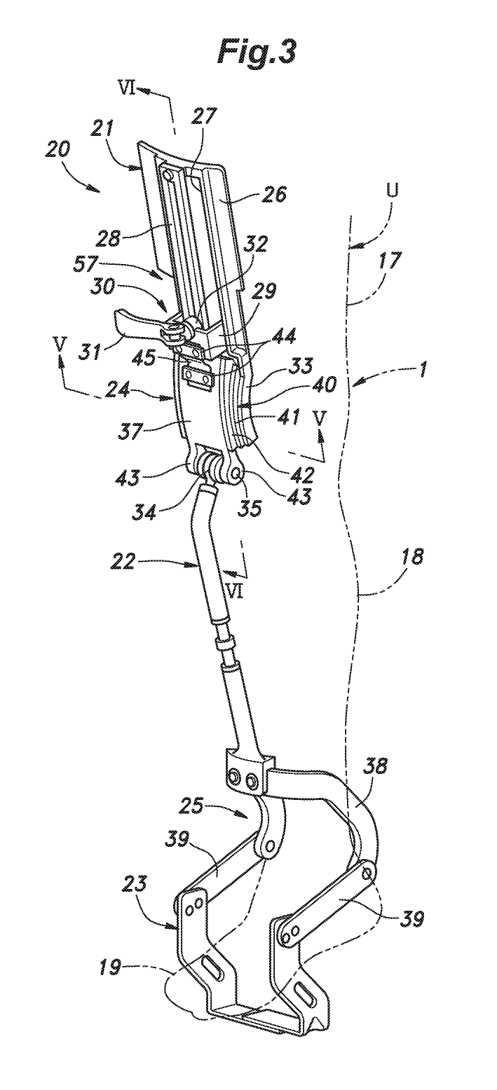

FIG. 3 is a perspective view of a right leg portion of the motion assist device 20 employing the knee joint mechanism 24 of the embodiment, where the knee joint mechanism 24 is shown in a contracted state corresponding to an extended state of the knee joint 1. The motion assist device 20 is a device for assisting motion of a body of the user U, and may be configured to assist walking motion of the legs of the user U or to assist bending and stretching (flexing and extending) motion of the legs of the user U, for example. The motion assist device 20 is configured to contact the ground when fitted on the leg of the user U such that the user U does not have to bear the entire weight of the device.

The motion assist device 20 includes, as main components thereof, a thigh link 21 configured to be disposed in front of a thigh 17 of the user U to extend vertically along the thigh 17, a lower leg link 22 configured to be disposed in front of a front portion of a lower leg 18 of the user U to extend vertically along the lower leg 18, and a foot holding member 23 configured to hold a foot 19 of the user U. The thigh link 21 and the lower leg link 22 are connected to each other by the knee joint mechanism 24, and the lower leg link 22 and the foot holding member 23 are connected to each other by a foot joint mechanism 25.

As described above, the axis of motion 1X of the knee joint 1 of the user U extends laterally, and in the basic limb position, the knee joint 1 is in a fully extended state with the lower leg 18 extending straight relative to the thigh 17, and when the knee joint 1 is flexed, the lower leg 18 is rotated rearward relative to the thigh 17 about the knee joint 1. Namely, with respect to the knee joint 1, the front side is an extension side, and the rear side is a flexion side. Therefore, the thigh link 21, the knee joint mechanism 24, and the lower leg link 22 are arranged on the extension side of the thigh 17, the knee joint 1, and the lower leg 18, respectively.

The thigh link 21 is formed by machining metallic material such as aluminum, and has a thigh link main body 26 having a curved cross-sectional shape conforming to the front surface of the thigh 17. On a front surface of the thigh link main body 26 is provided a pair of left and right first guide rails 27 extending linearly in the vertical direction. Further, at a portion of the front surface of the thigh link main body 26 between the left and right first guide rails 27 is provided a second guide rail 28 extending linearly in the vertical direction. The first guide rails 27 are slidably engaged with a slider 29 having a channel-like shape opening rearward.

The slider 29 is provided with a position adjustment member 30 capable of adjusting the position of the slider 29 on the first guide rails 27. The position adjustment member 30 includes an operation member 31 to be operated by the user and a locking member 32 configured to selectively engage with the second guide rail 28. The locking member 32 can be switched by an operation of the operation member 31 between an unlock state in which the locking member 32 is disengaged from the second guide rail 28 to permit sliding of the slider 29 relative to the second guide rail 28 (and hence the first guide rails 27) and a lock state in which the locking member 32 engages with the second guide rail 28 to prevent the slider 29 from sliding relative to the second guide rail 28 (and hence the first guide rails 27). Namely, when the locking member 32 is in the unlock state, the slider 29 can slide along the first guide rails 27, and when the locking member 32 is in the lock state, the slider 29 is fixed to the first guide rails 27.

The lower end of the thigh link 21 is connected to a first slide member 33 which is a part of the knee joint mechanism 24. In this embodiment, the first slide member 33 is formed integrally with the thigh link 21, and thus, fixed to the thigh link 21.

The lower leg link 22 is formed of properly processed bar-shaped and/or pipe-shaped members made of metal such as aluminum. The lower leg link 22 is provided at an upper end thereof with a plate-shaped upper connecting portion 34 having a laterally extending through-hole formed therein. The lower leg link 22 is pivotably connected to a second slide member 37, which is part of the knee joint mechanism 24, via a connecting pin 35 which extends laterally to pass through the through-hole of the upper connecting portion 34. The knee joint mechanism 24 including the first slide member 33 and the second slide member 37 will be described in detail later.

The lower end of the lower leg link 22 is connected to an upper member 38 constituting an upper part of the foot joint mechanism 25 and forming a hinge mechanism. The upper member 38 is formed by processing a metallic plate, such as that made of aluminum, and has a U-shape in plan view to surround a lower front part of the lower leg 18 with the left and right rear ends of the upper member 38 being placed on either lateral side of the foot joint. The left and right rear ends of the upper member 38 are pivotably connected to left and right lower members 39, respectively, where the lower members 39 constitute a lower part of the foot joint mechanism 25. Specifically, the rear ends of the lower members 39 are pivotably supported by the rear ends of the upper member 38 such that the pivot axis resides on the plantar/dorsi flexion axis of the foot joint.

The foot holding member 23 is connected to the front ends of the lower members 39. The foot holding member 23 is a part of the motion assist device to be in contact with the ground, and is configured to hold the foot 19 of the user U placed on a foot placement surface thereof. The foot holding member 23 can pivot about the plantar/dorsi flexion axis owing to the lower members 39 of the foot joint mechanism 25 pivotably connected to the upper member 38.

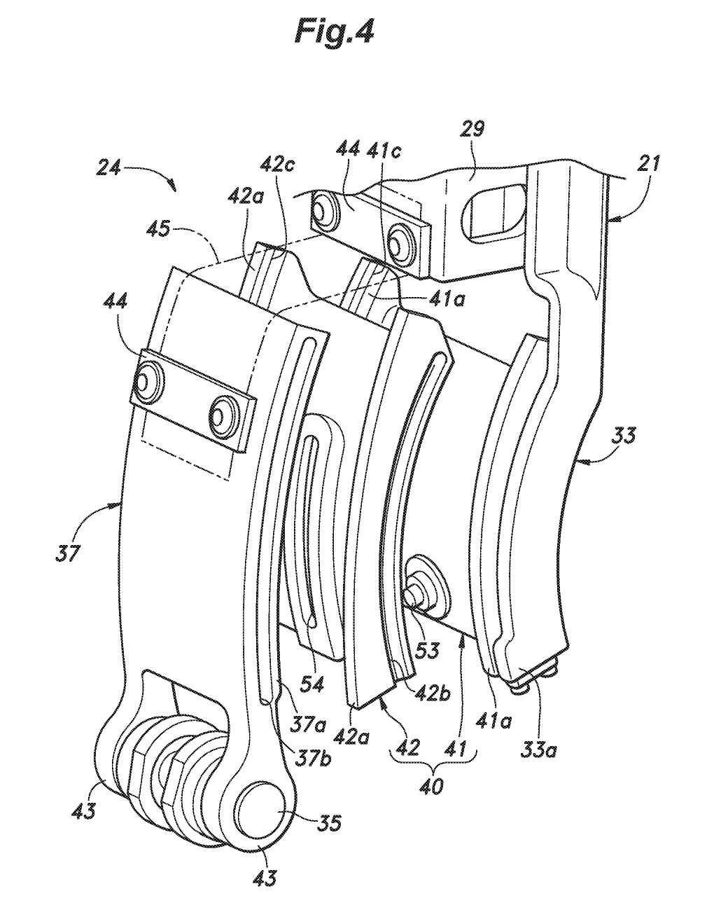

FIG. 4 is an exploded perspective view of the knee joint mechanism 24 shown in FIG. 3. As shown in FIG. 4, the knee joint mechanism 24 includes, in addition to the aforementioned first slide member 33 and second slide member 37, an intermediate slide member 40 including a first intermediate slide member 41 and a second intermediate slide member 42 and interposed between the first slide member 33 and the second slide member 37. In the illustrated embodiment, the second slide member 37 is disposed in front of the first slide member 33. The first intermediate slide member 41 is disposed on a side of the first slide member 33 while the second intermediate slide member 42 is disposed on a side of the second slide member 37 (namely, the first intermediate slide member 41 is disposed rearward of the second intermediate slide member 42). Each of the first slide member 33, the second slide member 37, the first intermediate slide member 41, and the second intermediate slide member 42 is formed by processing a metallic plate, such as that made of aluminum, and extends vertically in a curved manner so as to be convex toward the front (or away from the knee joint 1) in side view. The slide members 33, 37, 41, and 42 have pairs of left and right side walls 33a, 37a, 41a, 42a, respectively, extending along either side edge thereof.

The second slide member 37 is integrally formed at a lower end thereof with left and right pin supporting portions 43 that project downward. The pin supporting portions 43 support the connecting pin 35 for pivotably supporting the lower leg link 22.

A pair of fixing members 44 are detachably mounted on the front face of the slider 29 provided in the thigh link 21 and the front face of the second slide member 37, respectively, to fix an elastic member 45 shown by imaginary lines in FIG. 4. The two fixing members 44 fix the upper and lower ends of the elastic member 45 to the slider 29 and the second slide member 37, respectively, whereby the slider 29 and the second slide member 37 are connected to each other via the elastic member 45.

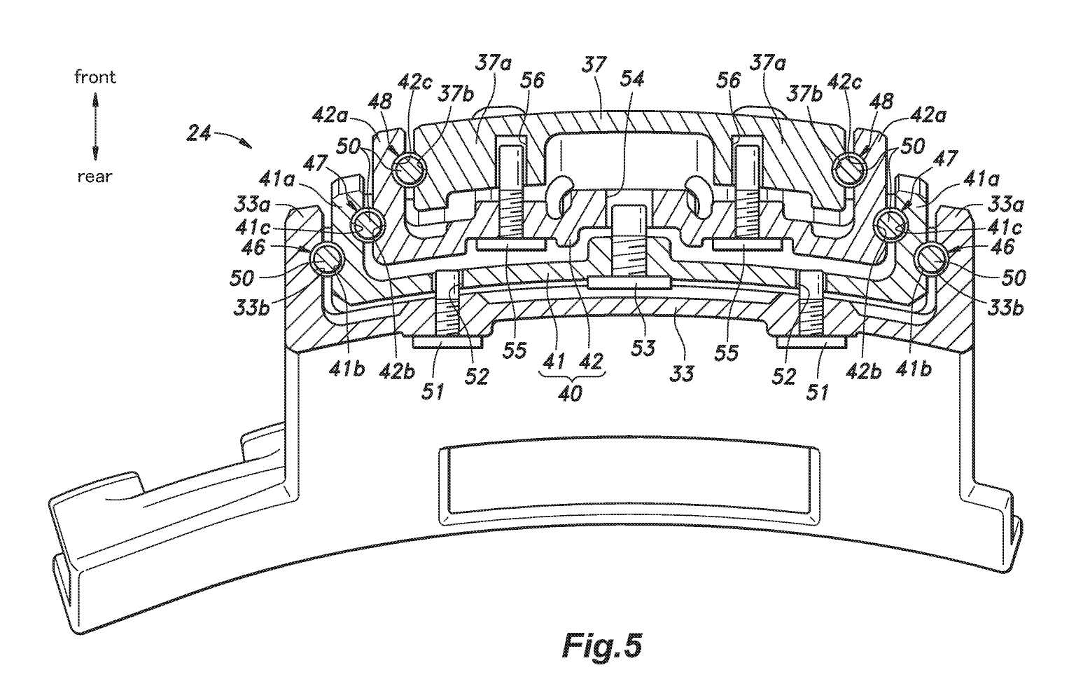

FIG. 5 is a sectional view taken along line V-V in FIG. 3. As shown in FIGS. 4 and 5, the left and right side walls 33a, 41a, 42a of the first slide member 33, the first intermediate slide member 41, and the second intermediate slide member 42 are each formed to project forward. On the other hand, the left and right side walls 37a of the second slide member 37 are formed to project rearward. The first intermediate slide member 41 is positioned inside the left and right side walls 33a of the first slide member 33, the second intermediate slide member 42 is positioned inside the left and right side walls 41a of the first intermediate slide member 41, and the second slide member 37 is positioned inside the left and right side walls 42a of the second intermediate slide member 42.

Inner surfaces of the left and right side walls 33a of the first slide member 33 are formed with left and right first slide tracks 33b, respectively, where each first slide track 33b is defined by a guide groove curved to have an arc shape that is convex toward the front in side view. Outer surfaces of the left and right side walls 41a of the first intermediate slide member 41 are formed with left and right first corresponding slide tracks 41b, respectively, which are configured to oppose and cooperate with the first slide tracks 33b, where each first corresponding slide track 41b is defined by a guide groove curved to have an arc shape that is convex toward the front in side view in correspondence with the opposing first slide track 33b. Each pair of the first slide track 33b and the first corresponding slide track 41b in cooperation define a first guide groove 46 having a circular cross-section. A plurality of steel balls 50 (bearing balls) are inserted in each first guide groove 46, such that the first slide member 33 and the first intermediate slide member 41 can slide relative to each other along the first guide grooves 46 via the steel balls 50.

Inner surfaces of the left and right side walls 41a of the first intermediate slide member 41 are formed with left and right third slide tracks 41c, respectively, where each third slide track 41c is defined by a guide groove curved to have an arc shape that is convex toward the front in side view. Outer surfaces of the left and right side walls 42a of the second intermediate slide member 42 are formed with left and right third corresponding slide tracks 42b, respectively, which are configured to oppose and cooperate with the third slide tracks 41c, where each third corresponding slide tracks 42b is defined by a guide groove curved to have an arc shape that is convex toward the front in side view in correspondence with the opposing third slide track 41c. Each pair of the third slide track 41c and the third corresponding slide track 42b in cooperation define a third guide groove 47 having a circular cross-section. The third guide grooves 47 are located slightly more forward than the first guide grooves 46. A plurality of steel balls 50 are inserted in each third guide groove 47, such that the first intermediate slide member 41 and the second intermediate slide member 42 can slide relative to each other along the third guide grooves 47 via the steel balls 50.

Inner surfaces of the left and right side walls 42a of the second intermediate slide member 42 are formed with left and right second slide tracks 42c, respectively, where each second slide track 42c is defined by a guide groove curved to have an arc shape that is convex toward the front in side view. Outer surfaces of the left and right side walls 37a of the second slide member 37 are formed with left and right second corresponding slide tracks 37b, respectively, which are configured to oppose and cooperate with the second slide tracks 42c, where each second corresponding slide track 37b is defined by a guide groove curved to have an arc shape convex toward the front in side view in correspondence with the opposing second slide track 42c. Each pair of the second slide track 42c and the second corresponding slide track 37b in cooperation define a second guide groove 48 having a circular cross-section. The second guide grooves 48 are located slightly more forward than the third guide grooves 47. A plurality of steel balls 50 are inserted in each second guide groove 48, such that the second intermediate slide member 42 and the second slide member 37 can slide relative to each other along the second guide grooves 48 via the steel balls 50.

The first slide member 33 has two first pin members 51 attached thereto from the rear side at two laterally spaced apart positions. Each first pin member 51 has a head and a shaft extending from the head and including a base end portion formed with male thread and a tip end portion formed with a smooth surface. With the threaded base end portion of the shaft engaging with a corresponding female threaded hole formed in the first slide member 33, each first pin member 51 is fixed to the first slide member 33 such that the smooth tip end portion of the shaft thereof projects from the front face of the first slide member 33, such that each first pin member 51 serves as a slide pin. The first intermediate slide member 41 is provided with two first guide slots 52 at positions corresponding to the two first pin members 51 to receive the shafts of the respective first pin members 51 therein, such that sliding of the first intermediate slide member 41 relative to the first slide member 33 is guided by the two first pin members 51.

The first intermediate slide member 41 has a single third pin member 53 attached thereto from the rear side at a laterally central position. The third pin member 53 has a structure similar to that of the first pin member 51, and with the threaded base end portion of the shaft thereof engaging with a corresponding female threaded hole formed in the first intermediate slide member 41, the third pin member 53 is fixed to the first intermediate slide member 41 such that the smooth tip end portion of the shaft projects from the front face of the first intermediate slide member 41. The second intermediate slide member 42 is provided with a single third guide slot 54 at a laterally central position corresponding to the third pin member 53 to receive the shaft of the third pin member 53, such that sliding of the second intermediate slide member 42 relative to the first intermediate slide member 41 is guided by the third pin member 53.

The second intermediate slide member 42 has two second pin members 55 attached thereto from the rear side at two laterally spaced apart positions more inward than the first pin members 51. Each second pin member 55 has a structure similar to that of the first pin member 51, and with the threaded base end portion of the shaft thereof engaging with a corresponding female threaded hole formed in the second intermediate slide member 42, each second pin member 55 is fixed to the second intermediate slide member 42 such that the smooth tip end portion of the shaft thereof projects from the front face of the second intermediate slide member 42. The second slide member 37 is provided with two second guide slots 56 at positions corresponding to the two second pin members 55 to receive the shafts of the respective second pin members 55, such that sliding of the second slide member 37 relative to the second intermediate slide member 42 is guided by the two second pin members 55.

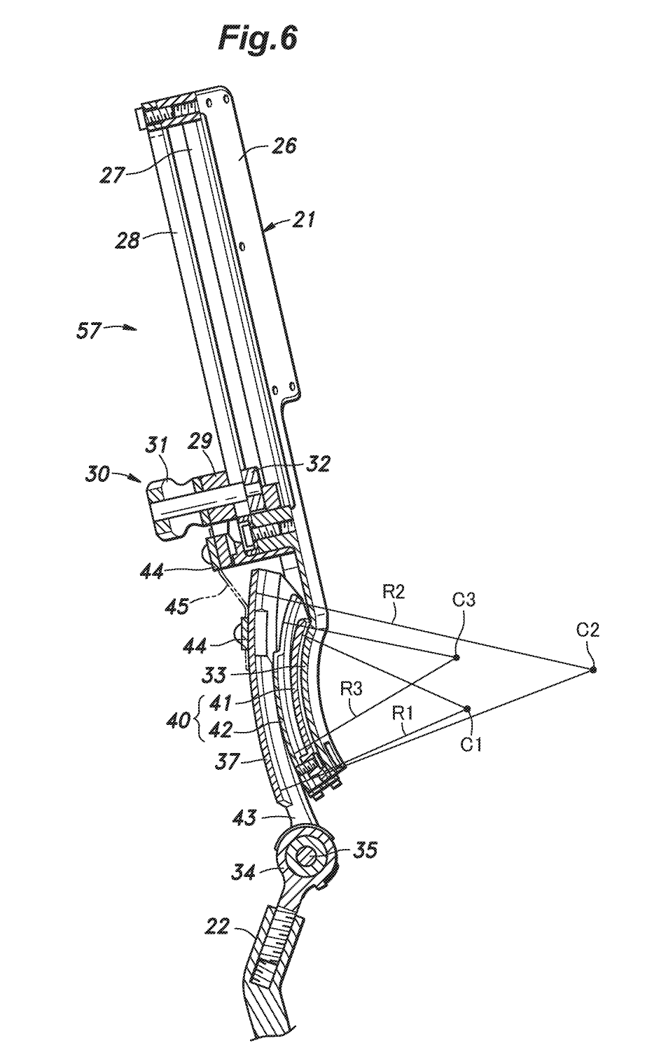

FIG. 6 is a sectional view taken along line VI-VI in FIG. 3. In FIG. 6, the knee joint mechanism 24 is shown in the contracted state, in which a substantial part of each of the slide members 33, 41, 42, and 37 overlaps with the adjoining slide member(s). As shown in FIG. 6, the center of curvature C1 of the first slide tracks 33b, the center of curvature C2 of the second slide tracks 42c, and the center of curvature C3 of the third slide tracks 41c are different from each other. Specifically, the center of curvature C1 of the first slide tracks 33b is located at a relatively forward position and at the lowermost position among the three centers of curvature, the center of curvature C3 of the third slide tracks 41c is located at a relatively forward and relatively high position, and the center of curvature C2 of the second slide tracks 42c is located at a relatively high position and at the most rearward position among the three centers of curvature. In addition, the radius of curvature R1 of the first slide tracks 33b, the radius of curvature R2 of the second slide tracks 42c, and the radius of curvature R3 of the third slide tracks 41c are different from each other. In the illustrated embodiment, the radius of curvature R1 of the first slide tracks 33b constituting the first guide grooves 46 (FIG. 5), which is located at the most rearward position (namely, closest to the knee joint 1) among the three guide grooves 46, 47, and 48, is the smallest of the three radii of curvature. The radius of curvature R2 of the second slide tracks 42c is the largest of the three radii of curvature as the second guide grooves 48 (FIG. 6) formed by the second slide tracks 42c are located at the most forward position among the three guide grooves 46, 47, and 48, and the center of curvature C2 of the second slide tracks 42c is located at the most rearward position among the three centers of curvature. The radius of curvature R3 of the third slide tracks 41c has a value intermediate between the radius of curvature R1 of the first slide tracks 33b and the radius of curvature R2 of the second slide tracks 42c.

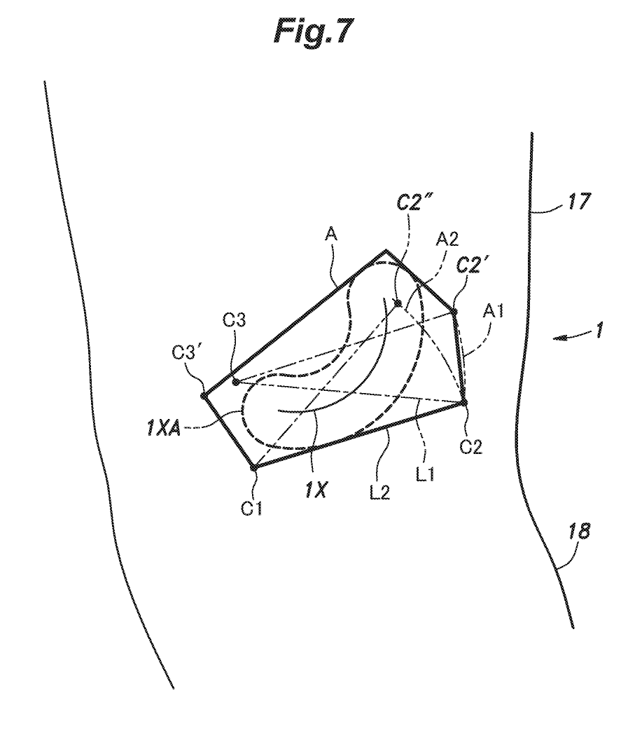

FIG. 7 is a schematic inner side view of the knee joint 1 of the right leg showing an area A in which an instant center of rotation of the knee joint mechanism 24 may be present. Since the second slide member 37 is slidable relative to the second intermediate slide member 42 along the second slide tracks 42c, the second slide member 37 is rotatable (pivotable) about the center of curvature C2 of the second slide tracks 42c. The second slide member 37 is also slidable, together with the second intermediate slide member 42, along the third slide tracks 41c, and hence is rotatable about the center of curvature C3 of the third slide tracks 41c. Therefore, in the state shown in FIG. 6 (namely, in the contracted state), if the sliding of the second intermediate slide member 42 (together with the second slide member 37) along the third slide tracks 41c and the sliding of the second slide member 37 along the second slide tracks 42c take place simultaneously, the second slide member 37 rotates about a point on a line segment L1 connecting the center of curvature C2 of the second slide tracks 42c and the center of curvature C3 of the third slide tracks 41c to each other, where the position of the point of rotation on the line segment L1 is determined according to a ratio between the two sliding motions. Namely, an instant center of rotation of the second slide member 37 in such a situation is on the line segment L1. It is to be noted that as the second intermediate slide member 42 slides along the third slide tracks 41c (namely, pivots about the center of curvature C3 of the third slide tracks 41c) from the state shown in FIG. 6, the center of curvature C2 of the second slide tracks 42c rotates about the center of curvature C3 of the third slide tracks 41c to a point C2′ shown in FIG. 7 together with the line segment L1, whereby a fan-shaped area A1 is defined by the points C3, C2 and C2′ in accordance with the slidable range of the second intermediate slide member 42 along the third slide tracks 41c. Thus, in this case, the instant center of rotation of the second slide member 37 may be located in or is moveable within the fan-shaped area A1, which is defined by the rotation of the line segment L1 about the center of curvature C3 of the third slide tracks 41c.

In addition, the second slide member 37 is also slidable, together with the first and second intermediate slide members 41, 42 (namely, together with the intermediate slide member 40), along the first slide tracks 33b, and hence, is rotatable about the center of curvature C1 of the first slide tracks 33b. Therefore, in the state shown in FIG. 6 (namely, in the contracted state), if the sliding of the intermediate slide member 40 (together with the second slide member 37) along the first slide tracks 33b and the sliding of the second slide member 37 along the second slide tracks 42c take place simultaneously, the second slide member 37 rotates about a point on a line segment L2 connecting the center of curvature C2 of the second slide tracks 42c and the center of curvature C1 of the first slide tracks 33b to each other, where the position of the point of rotation on the line segment L2 is determined according to a ratio between the two sliding motions. Namely, the instant center of rotation of the second slide member 37 in such a situation is on the line segment L2. It is to be noted that as the intermediate slide member 40 (more specifically, the first intermediate slide member 41) slides along the first slide tracks 33b (namely, pivots about the center of curvature C1 of the first slide tracks 33b) from the state shown in FIG. 6, the center of curvature C2 of the second slide tracks 42c rotates about the center of curvature C1 of the first slide tracks 33b to a point C2″ shown in FIG. 7 together with the line segment L2, whereby a fan-shaped area A2 is defined by the points C1, C2 and C2″ in accordance with the slidable range of the intermediate slide member 40 (more specifically, the first intermediate slide member 41) along the first slide tracks 33b. Thus, in this case, the instant center of rotation of the second slide member 37 may be located in or is moveable within the fan-shaped area A2 defined by the rotation of the line segment L2 about the center of curvature C1 of the first slide tracks 33b.

Further, when the intermediate slide member 40 (more specifically, the first intermediate slide member 41) rotates about the center of curvature C1 of the first intermediate slide member 41 from the state shown in FIG. 6, the center of curvature C3 of the third slide tracks 41c provided in the second intermediate slide member 42 of the intermediate slide member 40 also rotates about the center of curvature C1 of the first intermediate slide member 41 to a point C3′ shown in FIG. 7, and another fan-shaped area (not shown in FIG. 7) is defined by the points C1, C3 and C3′, in which the instant center of rotation of the second slide member 37 may be located. Moreover, because the second slide member 37 is rotatable about the point C3′ together with the second intermediate slide member 42, yet another fan-shaped area (not shown in FIG. 7) in which the instant center of rotation of the second slide member 37 may be located can be defined by rotating a line segment (not shown in FIG. 7) connecting the point C3′ and the point C2″ about the point C3′. By combining these fan-shaped areas, an area A surrounded by a thick solid line in FIG. 7 is obtained as an area in which the instant center of rotation of the second slide member 37 relative to the first slide member 33 (which may also be referred to as the instant center of rotation of the joint angle mechanism 24) may be located. This area A is defined to include a trajectory of the axis of motion 1X of the knee joint 1 of the user U during the flexion/extension of the knee joint 1 indicated by a solid curve in FIG. 7. More specifically, the slide tracks of the slide members of the knee joint mechanism 24 are designed such that the area A in which the instant center of rotation of the second slide member 37 relative to the first slide member 33 may be located includes the trajectory of the axis of motion 1X of the knee joint 1.

Incidentally, the manner of movement of the axis of motion 1X of the knee joint 1 may vary depending on the size and shape of the bones, which differ from one person to another. Namely, due to individual differences, trajectories of the axis of motion 1X of the knee joint 1 of various users may be distributed over a certain area 1XA as shown by broken lines in FIG. 7. In the present embodiment, the area A of the instant center of rotation of the knee joint mechanism 24 (the instant center of rotation of the second slide member 37 relative to the first slide member 33) is defined to include a substantially entire part of the area 1XA (shown in broken lines) in which the trajectories of the axis of motion 1X of the knee joint 1 of multiple users may be present, so that the knee joint mechanism 24 can be used commonly by multiple users.

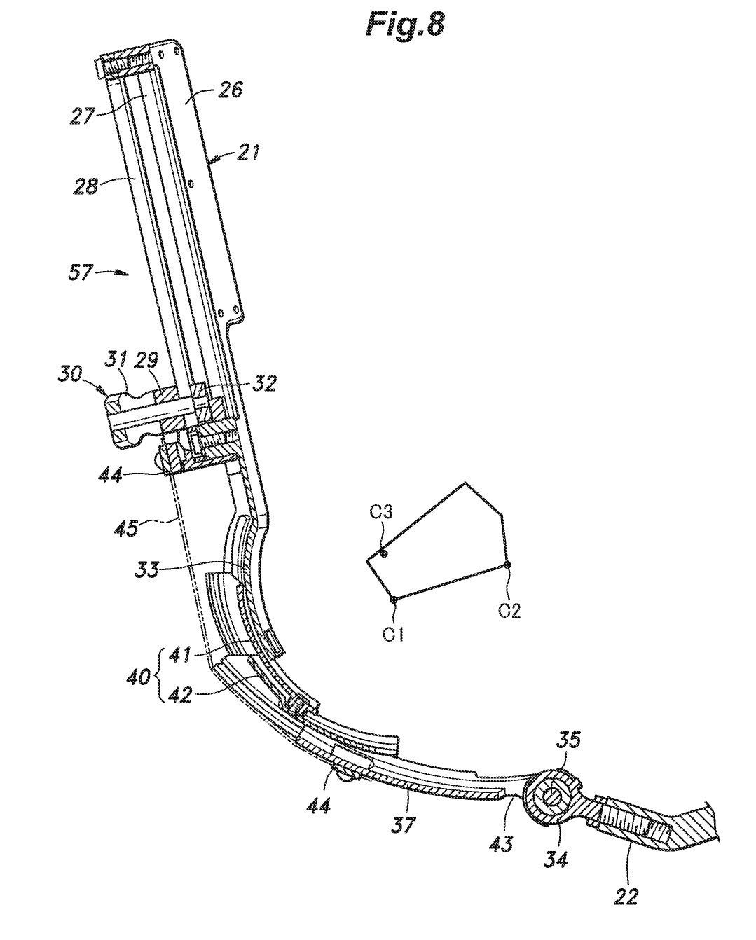

FIG. 8 is a sectional view similar to FIG. 6 and showing the knee joint mechanism 24 in a deployed state corresponding to a flexed state of the knee joint 1. In the deployed state of the knee joint mechanism 24, a smaller part of each of the slide members 33, 41, 42, and 37 overlaps with the adjoining slide member(s) compared to when in the contracted state, so that the length of the knee joint mechanism 24 is longer and the distance between the thigh link 21 and the lower leg link 22 connected by the knee joint mechanism 24 is larger than in the contracted state. When the user U flexes the knee joint 1 (pivots the lower leg 18 rearward), the foot holding member 23 holding the foot 19 joined to the lower end of the lower leg 18 is moved rearward, and the lower leg link 22 connected to the foot holding member 23 pivots rearward together with the lower leg 18, with the adjoining slide members of the knee joint mechanism 24 sliding relative to each other.

At this time, the elastic member 45, which is disposed in front of the knee joint 1 and connecting the slider 29 locked on the thigh link 21 and the second slide member 37 connected to the lower leg link 22, is expanded by the rearward pivoting of the lower leg link 22, and produces a moment to urge the flexed knee joint 1 toward the extended state. Namely, the elastic member 45 serves as a tensile elastic member that, when the knee joint 1 is flexed, produces an auxiliary moment on the first link (thigh link 21) and the second link (lower leg link 22) in a direction to extend the knee joint 1. As the bending angle of the knee joint 1 increases, the elastic member 45 is expanded more. Hence, the more the bending angle increases, the larger auxiliary moment for extending the knee joint 1 is imparted on the knee joint 1.

In addition, as shown in FIGS. 3 and 4, the position of the slider 29 to which the elastic member 45 is attached is adjustable by the position adjustment member 30 in the direction of extension of the second guide rail 28, and this allows the tensile force of the elastic member 45 in the basic limb position (or when the knee joint 1 is in an extended state) to be adjusted. Namely, the second guide rail 28, the slider 29, and the position adjustment member 30 constitute a moment adjustment mechanism 57 for adjusting the moment produced by the elastic member 45 by adjusting the connection positon of the elastic member 45 to the thigh link 21 in the direction of expansion of the elastic member 45.

In the following, technical advantages of the knee joint mechanism 24 having the above structure will be described.

As shown in FIGS. 6 and 7, the knee joint mechanism 24 includes the first slide member 33, the intermediate slide member 40, and the second slide member 37, where the first slide tracks 33b, along which the intermediate slide member 40 is slidable relative to the first slide member 33, and the second slide tracks 42c, along which the second slide member 37 is slidable relative to the intermediate slide member 40, have different centers of curvature C1, C2. Therefore, the instant center of rotation of the second slide member 37 relative to the first slide member 33 is located on the line segment L2 that connects the center of curvature C1 of the first slide tracks 33b and the center of curvature C2 of the second slide tracks 42c, and as the intermediate slide member 40 slides along the first slide tracks 33b (namely, pivots about the center of curvature C1 of the first slide tracks 33b), the line segment L2 rotates about the center of curvature C1 of the first slide tracks 33b together with the center of curvature C2 of the second slide tracks 42c, to define the fan-shaped area A2. Namely, the instant center of rotation of the second slide member 37 may be located in or is moveable within the fan-shaped area A2 defined by the rotation of the line segment L2 about the center of curvature C1 of the first slide tracks 33b. In other words, the instant center of rotation of the lower leg link 22 (which is connected to the second slide member 37) relative to the thigh link 21 is moveable within the fan-shaped area A2, and this reduces the discomfort caused to the user U when the knee joint 1 is moved.

As shown in FIGS. 4 and 5, the intermediate slide member 40 includes the first intermediate slide member 41 slidably connected to the first slide member 33 via the first slide tracks 33b, and the second intermediate slide member 42 slidably connected to the second slide member 37 via the second slide tracks 42c and to the first intermediate slide member 41 via the third slide tracks 41c, where the center of curvature C3 of the third slide tracks 41c is different from the center of curvature C1 of the first slide tracks 33b and the center of curvature C2 of the second slide tracks 42c. Thereby, the area in which the instant center of rotation of the second slide member 37 relative to the first slide member 33 may be located can be widened from the fan-shaped area A2 to the area A as shown in FIG. 7.

Further, the radius of curvature R2 of the second slide tracks 42c is different from the radius of curvature R1 of the first slide tracks 33b. Thereby, the length of the line segment L2 connecting the center of curvature C1 of the first slide tracks 33b and the center of curvature C2 of the second slide tracks 42c to each other can be adjusted as desired, whereby the fan-shaped area A2 defined by rotation of the line segment L2 about the center of curvature C1 can be given a desired size.

The radius of curvature R3 of the third slide tracks 41c differs from the radius of curvature R1 of the first slide tracks 33b and the radius of curvature R2 of the second slide tracks 42c. Thereby, the length of the line segment L1 connecting the center of curvature C3 of the third slide tracks 41c and the center of curvature C2 of the second slide tracks 42c to each other can be adjusted as desired, whereby the fan-shaped area A1 defined by rotation of the line segment L1 about the center of curvature C3 can be given a desired size.

As shown in FIG. 3, the second slide member 37 is connected to the lower leg link 22 so as to be pivotable about a pivot axis to be parallel with the axis of motion 1X of the knee joint 1 (FIG. 2), and this enables the relative angle between the thigh link 21 and the lower leg link 22 to be adjusted to be in conformity with the angle of the knee joint 1 of the user U in a basic (natural) posture. It is to be noted that instead of or in addition to the second slide member 37, the first slide member 33 may be connected to the thigh link 21 so as to be pivotable about a pivot axis to be parallel with the axis of motion 1X of the knee joint 1.

As shown in FIG. 8, the knee joint mechanism 24 is provided with the elastic member 45 connected to the thigh link 21 and the second slide member 37 such that, when expanded, the elastic member 45 produces a moment, resulting from a tensile force, on the thigh link 21 and the second slide member 37 (and hence, on the lower leg link 22 connected to the second slide member 37) in a direction to extend the knee joint 1 or in a direction to bring the thigh link 21 and the lower leg link 22 closer to each other. Thus, a moment (tensile force) for extending the knee joint 1 can be produced with a simple structure.

As shown in FIG. 4, the knee joint mechanism 24 further includes the moment adjustment mechanism 57 configured to adjust the moment produced from the elastic member 45 by adjusting the connection position of the elastic member 45 to the thigh link 21 in the direction of expansion of the elastic member 45, and this enables an amount of assisting moment appropriate to each user to be applied on the thigh link 21 and the lower leg link 22.

In the illustrated embodiment, the joint mechanism according to the present invention is embodied as the knee joint mechanism 24. Thereby, when the user U flexes/extends the knee joint 1, the instant center of rotation of the second slide member 37 relative to the first slide member 33 can move in accordance with movement of the axis of motion 1X of the knee joint 1 as shown in FIG. 7, and therefore, the discomfort caused to the user U during flexing/extending motion of the knee joint 1 can be reduced.

As shown in FIG. 7, the area A in which the instant center of rotation of the second slide member 37 relative to the first slide member 33 may be located includes the trajectory of the axis of motion 1X of the knee joint 1 of the user U, whereby it is ensured that the knee joint mechanism 24 can follow the movement of the axis of motion 1X of the knee joint 1 of the user U to thereby reduce the discomfort caused to the user U during the flexing/extending motion of the knee joint 1. It is also possible to define the area A in which the instant center of rotation of the second slide member 37 relative to the first slide member 33 may be located such that the area A includes trajectories of the axis of motion 1X of the knee joint 1 of a plurality of users, such that the knee joint mechanism 24 can be commonly used by the plurality of users.

A preferred embodiment of the present invention has been described in the foregoing, but the present invention is not limited to the above-described embodiment and various modifications and alterations are possible. For example, in the above embodiment, the adjoining slide members are configured to slide via the steel balls 50, but the adjoining slide members may be configured to slide relative to each other via a guide rail mechanism including a member having a projection and a member having a recess to slidably receive the projection. Further, in the foregoing the embodiment, the second slide member 37 and the lower leg link 22 are rotatably connected to each other, but they may be fixedly connected to each other or may be formed integrally to each other. Also, the motion assist device including the joint mechanism of the present invention may be fitted on an elbow of a user instead of the knee to assist lifting or pushing of an object by the arm of the user. Besides, the concrete structure, number, angle, material, etc. of the structural elements of the present invention shown in the above embodiment may be changed appropriately within the scope of the present invention defined by the claims. Further, not all of the structural elements shown in the above embodiment are necessarily indispensable, and they may be selectively adopted as appropriate.

1. A joint mechanism pivotably connecting a first link and a second link respectively to be fitted on a first body part and a second body part of a user to assist motion of the user, the first and second body parts being connected to each other via a joint, the first link extending along an extension side of the first body part when fitted on the first body part, the second link extending along an extension side of the second body part when fitted on the second body part, the joint mechanism comprising:
a first slide member connected to the first link and provided with a first slide track having an arc shape that is convex away from the joint in side view;
an intermediate slide member provided with a first corresponding slide track configured to cooperate with the first slide track and a second slide track having an arc shape that is convex away from the joint in side view, and connected to the first slide member so as to be slidable along the first slide track; and
a second slide member provided with a second corresponding slide track configured to cooperate with the second slide track, connected to the intermediate slide member so as to be slidable along the second slide track, and connected to the second link,
wherein the first and second slide tracks have different centers of curvature from each other.

2. The joint mechanism according to claim 1, wherein the intermediate slide member comprises:
a first intermediate slide member provided with the first corresponding slide track and a third slide track having an arc shape that is convex away from the joint in side view, and connected to the first slide member so as to be slidable along the first slide track; and
a second intermediate slide member provided with the second slide track and a third corresponding slide track configured to cooperate with the third slide track, and connected to the first intermediate slide member so as to be slidable along the third slide track,
wherein the third slide track has a center of curvature different from the centers of curvature of the first slide track and the second slide track.

3. The joint mechanism according to claim 1, wherein the second slide track has a radius of curvature different from a radius of curvature of the first slide track.

4. The joint mechanism according to claim 2, wherein the third slide track has a radius of curvature different from at least one of a radius of curvature of the first slide track and a radius of curvature of the second slide track.

5. The joint mechanism according to claim 1, wherein at least one of the first slide member and the second slide member is connected to a corresponding one of the first link and the second link so as to be pivotable about a pivot axis to be parallel with an axis of motion of the joint.

6. The joint mechanism according to claim 1, further comprising an elastic member connected to the first link and one of the second link and the second slide member such that, when expanded, the elastic member produces a moment on the first link and the second link in a direction for extending the joint.

7. The joint mechanism according to claim 6, further comprising a moment adjustment mechanism configured to adjust the moment produced from the elastic member by adjusting a connection position of the elastic member to the first link in a direction of expansion of the elastic member.

8. The joint mechanism according to claim 1, wherein the first body part is a thigh, the second body part is a lower leg, and the joint is a knee joint, and
wherein the first slide member, the intermediate slide member, and the second slide member are configured to be arranged in front of the knee joint.

9. The joint mechanism according to claim 8, wherein the first and second slide tracks are configured such that an area in which an instant center of rotation of the second slide member relative to the first slide member may be located includes a trajectory of an axis of motion of the knee joint.
